(19) United States
(12) Patent Application Publication
(10) Pub. No.: US 2015/0320691 A1
(43) Pub. Date: Nov. 12, 2015

(54) USE OF MAGNESIUM STEARATE IN DRY POWDER FORMULATIONS FOR INHALATION

(71) Applicant: CHIESI FARMACEUTICI S.p.A., Parma (IT)

(72) Inventors: Daniela COCCONI, Parma (IT); Massimiliano Dagli ALBERI, Parma (IT); Andrea BUSCA, Parma (IT); Francesca SCHIARETTI, Parma (IT)

(73) Assignee: CHIESI FARMACEUTICI S.p.A., Parma (IT)

(21) Appl. No.: 14/801,882

(22) Filed: Jul. 17, 2015

Related U.S. Application Data
(63) Continuation of application No. 13/239,903, filed on Sep. 22, 2011.

(30) Foreign Application Priority Data
Sep. 30, 2010 (EP) 10183018.0

(51) Int. Cl. A61K 9/50; A61K 47/26; A61K 47/12; A61K 31/439

(52) U.S. Cl. CPC A61K 9/5015; A61K 9/5089; A61K 31/439; A61K 47/26; A61K 47/12

(57) ABSTRACT

Addition of magnesium stearate to a powder formulation for inhalation comprising carrier particles and an active ingredient bearing a group susceptible to hydrolysis is useful for inhibiting or reducing the chemical degradation of the active ingredient.

### CROSS REFERENCES TO RELATED APPLICATIONS

This application is a continuation of European Patent Application No. 10183018.0, filed on Sep. 30, 2010, which is incorporated herein by reference in its entirety.

### BACKGROUND OF THE INVENTION

1. Field of the Invention

The present invention relates to carrier particles for use in dry powder formulations for inhalation, and formulations thereof. In particular, the present invention relates to the use of magnesium stearate in powder formulation for inhalation comprising carrier particles to inhibit or reduce chemical degradation of an active ingredient bearing a group susceptible to hydrolysis. The present invention also relates to methods for producing such carrier particles, pharmaceutical formulations which contain such carrier particles, and method of treating certain conditions by administering such a pharmaceutical formulation.

2. Discussion of the Background

Dry powder inhalation (DPI) drug therapy has been used for many years to treat respiratory conditions such as asthma, chronic obstructive pulmonary disease (COPD), and allergic rhinitis. Compared to oral drug intake, only relatively small doses are needed for effective therapy as first pass metabolism is significantly reduced. Such small doses reduce the body's exposure to the drug and minimize side effects. Systemic adverse effects are also reduced as topical lung delivery takes the drug directly to the site of action. Lower dosage regimens may also provide considerable cost savings, particularly where expensive therapeutic agents are concerned.

Dry powder forms are typically formulated by mixing the drug in micronized form with physiologically acceptable pharmacologically inert coarse carrier particles, giving rise to ordered mixture where the micronized active particles adhere to the surface of the carrier particles whilst in the inhaler device. During inhalation, the drug particles separate from the surface of carrier particles and penetrate into the lower lungs, while the larger carrier particles are mostly deposited in the oropharyngeal cavity.

To promote the release of the drug particles from the surface carrier particles and, hence, to increase the fraction of respirable particles, additives with lubricant or anti-adherent properties have been proposed in the art. A particularly useful additive has been found to be magnesium stearate.

The benefit of using magnesium stearate in dry powders is taught for example in U.S. Pat. No. 6,528,096, which is incorporated herein by reference in its entirety. Specifically, it teaches that magnesium stearate can be used to alter the surface properties of carrier particles and thereby improve the properties of dry powder formulations. This reference also reports an “advantageous relationship” between surface coating carrier particles with magnesium stearate and the fine particle fraction (respirable fraction) of the emitted dose.

Besides the delivered respirable fraction, another important requirement is that the active ingredient should be chemically stable in the dry powder pharmaceutical formulations on storage. In fact, it is known that active substances may demonstrate instability to one or more factors, i.e. heat, light or moisture, and various precautions must be taken in formulating and storing such substances to ensure that the pharmaceutical products remain in an acceptable condition for use over a reasonable period of time, such that they have an adequate shelf-life. In particular, active ingredients bearing certain groups such as a carbonate group, a carbamate group, and an ester group, in the presence of high temperature and/or percentage of moisture could give rise to degradation products occurring through hydrolysis pathway.

WO 00/28979, which is incorporated herein by reference in its entirety, describes the use of magnesium stearate in dry powder formulations for inhalation to improve resistance to moisture and to reduce the effect of penetrating moisture on the fine particle fraction, i.e. the respirable fraction of an inhaled formulation. Such interference with physical interactions between a carrier and a drug substance is distinct from chemical instability resulting from degradation.

WO 2005/004845, which is incorporated herein by reference in its entirety, discloses the use of magnesium stearate to inhibit or reduce chemical interaction between an active ingredient substance and a carrier in a solid pharmaceutical formulation, wherein the active ingredient substance is susceptible to chemical interaction with the carrier through Maillard reaction. Said reaction involves the formation of adducts between amines and reducing sugars such al lactose. Thus it is distinct from hydrolysis and concern drugs active ingredients bearing primary or secondary amino groups.

### SUMMARY OF THE INVENTION

Accordingly, it is one object of the present invention to provide novel carrier particles which are useful for carrying particles of an active ingredient which has a group susceptible to hydrolysis.

It is another object of the present invention to provide novel methods for preparing such carrier particle.

It is another object of the present invention to provide novel powder formulations for inhalation comprising carrier particles and an active ingredient bearing a group susceptible to hydrolysis selected from the group consisting of a carbonate group, a carbamate group, and an ester group.

It is another object of the present invention to provide novel methods for preparing such a powder formulation.

It is another object of the present invention to provide novel methods of treating and/or preventing certain conditions by administering such a formulation.

It is another object of the present invention to provide novel methods for stabilizing an active ingredient which is contained in a powder formulation and which bears a group susceptible to hydrolysis selected from the group consisting of a carbonate group, a carbamate group, and an ester group to inhibit or reduce chemical degradation of the active ingredient.

These and other objects, which will become apparent during the following detailed description, have been achieved by the inventors' discovery that including magnesium stearate in a powder formulation for inhalation comprising carrier particles and an active ingredient bearing a group susceptible to hydrolysis selected from the group consisting of a carbonate group, a carbamate group, and an ester group can inhibit or reduce chemical degradation of said active ingredient.

Thus, in a first aspect, the present invention provides the use of magnesium stearate in powder formulation for inhalation comprising carrier particles and an active ingredient bearing a group susceptible to hydrolysis selected from the group consisting of a carbonate group, a carbamate group, and an ester group to inhibit or reduce chemical degradation of said active ingredient.

Preferably, said magnesium stearate coats the surface of the carrier particles.

The chemical stability of the active ingredient in the powder formulation may thereby be improved.

In a second aspect, the present invention provides a method to inhibit or reduce chemical degradation of an active ingredient for inhalation bearing a group susceptible to hydrolysis selected from the group consisting of a carbonate group, a carbamate group, and an ester group, which comprises mixing the carrier particles with magnesium stearate.

In a third aspect, the present invention provides a dry powder pharmaceutical formulation for inhalation comprising (a) carrier particles, (b) magnesium stearate, and (c) an active ingredient belonging to the class of the antimuscarinic drug bearing a carbonate group.

In a fourth aspect, the present invention provides a dry powder inhaler filled with afore mentioned pharmaceutical formulation.

In another aspect, the present invention provides methods for the prophylaxis and/or treatment of certain conditions by administering an effective amount of such a pharmaceutical formulation.

The present inventors have found that, in the presence of magnesium stearate, active ingredients bearing chemical group prone to hydrolysis turn out to be more chemically stable upon storage in particular stressing conditions i.e., in the presence of high temperature and/or high percentage of moisture.

### BRIEF DESCRIPTION OF THE DRAWINGS

A more complete appreciation of the invention and many of the attendant advantages thereof will be readily obtained as the same become better understood by reference to the following detailed description when considered in connection with the accompanying drawings, wherein:

FIG. 1 is a sketch of the film forming process around a single carrier particles.

FIG. 2 is a plot of CHF 5551.02 content versus time (ambient=60% relative humidity, R.H.).

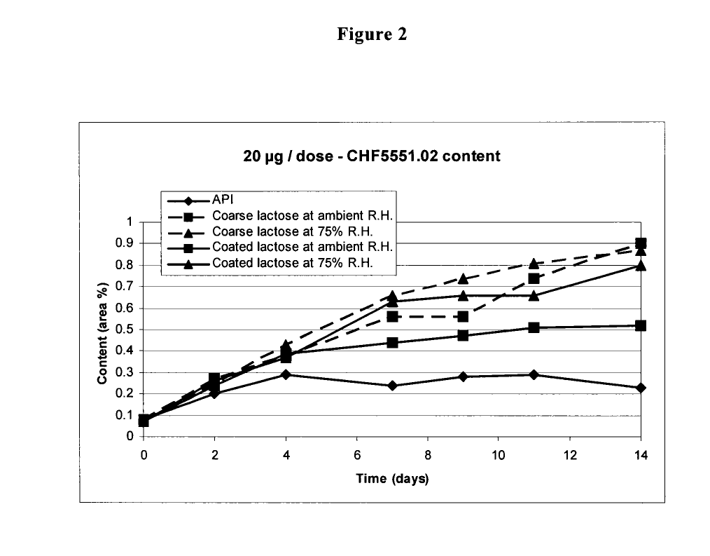

### DETAILED DESCRIPTION OF THE PREFERRED EMBODIMENTS

The terms “active drug,” “active ingredient,” “active,” “active substance,” and “active compound” are used synonymously.

The terms “muscarinic receptor antagonists,” “antimuscarinic drugs,” and “anticholinergic drugs” are used synonymously.

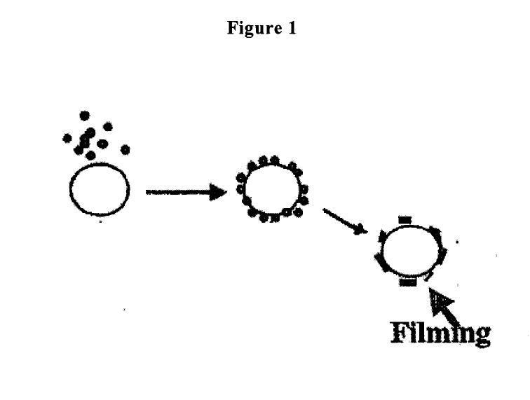

The term “coating” refers to the covering of the surface of the carrier particles by forming a (mono)molecluar film of magnesium stearate around said particles as reported in the sketch of FIG. 1.

The percentage of surface coating indicate the extent by which magnesium stearate coats the surface of all the carrier particles.

The expression “to inhibit or reduce chemical degradation of an active ingredient” means that, upon storage, degradation product(s) arising from hydrolysis of a group susceptible to hydrolytic cleavage are not formed or are formed in a lesser amount than the formulation comprising no magnesium stearate.

“Single dose” refers to the quantity of active ingredient administered at one time by inhalation upon actuation of an inhaler.

“Actuation” refers to the release of active ingredient from a device by a single activation (e.g. mechanical or breath).

The expression “chemically stable” refers to a formulation that meets the requirements of the ICH Guideline Q1A referring to “Stability Testing of new Active Substances (and Medicinal Products)”.

In general terms, the particle size of particles is quantified by measuring a characteristic equivalent sphere diameter, known as volume diameter, by laser diffraction.

The particle size can also be quantified by measuring the mass diameter by means of suitable known instruments, such as sieving.

The volume diameter (VD) is related to the mass diameter (MD) by the density of the particles (assuming a size independent density for the particles).

In the present application, the particle size interval is expressed in terms of mass diameter. The particle size distribution is generally expressed in terms of: i) the volume median diameter (VMD) or the mass median diameter (MMD) which corresponds to the diameter of 50 percent of the particles by volume or weight respectively, e.g. d(0.5); and ii) the mass or volume diameter of 10% and 90% of the particles, respectively, e.g. d(0.1) and d(0.9).

The expression “respirable fraction” refers to an index of the percentage of active particles which would reach the deep lungs in a patient.

The respirable fraction, also termed fine particle fraction, is commonly evaluated using suitable in vitro apparata such as Multistage Cascade Impactor or Multi Stage Liquid Impinger (MLSI) according to procedures reported in common Pharmacopoeias.

The present invention finds application in dry powder formulations for inhalation comprising carrier particles and at least one micronized active ingredient.

The carrier particles may be made of any physiologically acceptable pharmacologically-inert material or combination of materials suitable for inhalatory use. For example, the carrier particles may be composed of one or more materials selected from sugar alcohols; polyols, for example sorbitol, mannitol, and xylitol, and crystalline sugars, including monosaccharides and disaccharides; inorganic salts such as sodium chloride and calcium carbonate; organic salts such as sodium lactate; and other organic compounds such as urea, polysaccharides, for example starch and its derivatives; oligosaccharides, for example cyclodextrins and dextrins.

Advantageously, the carrier particles are made of a crystalline sugar, for example, a monosaccharide such as glucose or arabinose, or a disaccharide such as maltose, saccharose, dextrose or lactose. Preferably, the carrier particles are made of lactose, more preferably of alpha-lactose monohydrate.

Advantageously, said coarse carrier particles have a mass diameter (MD) of at least 20 microns, more advantageously greater that 50 microns. Preferably the MD is 50 microns to 1000 microns, more preferably 80 to 500 microns.

In certain embodiments of the invention, the MD is 90 to 150 microns.

In other embodiments, the MD is 150 to 400 microns, and preferably 210 to 355 microns.

The desired particle size may be obtained by sieving.

When their MD is 150 to 400 microns, the coarse carrier particles have preferably a relatively highly fissured surface, that is, on which there are clefts and valleys and other recessed regions, referred to herein collectively as fissures.

The “relatively highly fissured” coarse particles can be defined in terms of fissure index or rugosity coefficient as described in WO 01/78695 and WO 01/78693, which are incorporated herein by reference in their entireties, and they can be characterized according to the description therein reported.

Said coarse carrier particles may also be characterized in terms of tapped density or total intrusion volume measured as reported in WO 01/78695, which is incorporated herein by reference in its entirety.

The tapped density of the coarse carrier particles is advantageously less than 0.8 g/cm³, preferably 0.8 to 0.5 g/cm³.

The total intrusion volume is of at least 0.8 cm³ preferably at least 0.9 cm³.

The formulation may also comprise fine particles of a physiologically acceptable pharmacologically-inert material with a mass median diameter (MMD) equal to or less than 10 microns.

The percentage of said fine particles is advantageously 0.1 to 20% of the total amount of the formulation, preferably 5 to 15%.

Preferably, the coarse particles and the fine particles are constituted of the same physiologically acceptable pharmacologically-inert material.

The amount of magnesium stearate present in the formulation varies depending on both the dry powder inhaler and type the active ingredient. The skilled person, aware of the physical and chemical properties of the active ingredient and of the type of inhaler, for example single dose or multidose, will be able to select an appropriate amount.

Advantageously, the amount of magnesium stearate may be 0.05 to 1.5% by weight of the carrier. More advantageously it may be 0.1 to 1.0% by weight of the carrier.

In a preferred embodiment, the amount of magnesium stearate may be 0.15 to 0.5%, more preferably 0.2 to 0.4% w/w, while in other preferred embodiments it may be 0.5 to 1.5% by weight or 0.8 to 1.0% w/w.

Preferably, the carrier particles are subjected to coating with magnesium stearate particles until the extent of coating is of at least 10%, advantageously higher than 12%, preferably equal to or higher than 20%.

Coating could be obtained by mixing the carrier particles and the magnesium stearate particles according to the conditions disclosed in WO 00/53157, which is incorporated herein by reference in its entirety.

In particular embodiments, by applying the conditions disclosed in the co-pending application EP 10158951.3, which is incorporated herein by reference in its entirety, an extent of coating of more than 60%, advantageously higher than 70%, more advantageously of at least 80%, preferably higher than 85%, more preferably higher than 90%, even more preferably higher than 95% could be obtained.

If alpha-lactose monohydrate is used as a carrier, the extent to which magnesium stearate coats the surface of the carrier particles may be determined by first measuring the water contact angle, and then applying the equation known in the literature as Cassie or Cassie and Baxter, reported anyway as follows (Colombo I et al Il Farmaco 1984, 39(10), p. 338) (which is incorporated herein by reference in its entirety:

cos θₘᵢₓₜᵤᵣₑ=f_{MgSt} cos θ_{MgSt}+f_{lactose} cos θ_{lactose}

where f_{MgSt} and f_{lactose} are the surface area fractions of magnesium stearate and of lactose;

θ_{MgSt} is the water contact angle of magnesium stearate;

θ_{lactose} is the water contact angle of lactose

θₘᵢₓₜᵤᵣₑ are the experimental contact angle values.

The measure of the contact angle between a liquid and a solid surface is commonly used in the art for determining the wettability of solids. This approach is based on the capability of a liquid to spread spontaneously over the surface of a solid to reach a thermodynamic equilibrium.

For the purpose of the present invention, the contact angle may be determined with methods that are essentially based on goniometric measurement. These imply the direct observation of the angle formed between the solid substrate and the liquid under testing. It is therefore quite simple to carry out, being the only limitation related to possible bias stemming from intra-operator variability. It should be, however, underlined that this drawback can be overcome by adoption a fully automated procedure, such as a computer assisted image analysis.

A particularly useful approach is the sessile or static drop method which is typically carried out by depositing a liquid drop onto the surface of the powder in form of disc obtained by compaction (known as compressed powder disc method).

Typically, the procedure is carried out as follows:

The compressed disc is prepared by adding the sample into the die of a press and a compression force of 5 kN is applied for 3 minutes. Then the compressed disc is placed on a plate of a surface wettability tester and a water drop of about 10 μl is formed on the surface of the disc.

A suitable surface wettability tester is, for example, that available from Lorentzen & Wettre GmbH.

The pictures are taken with a videocamera and the water contact angles values are given by a computer assisting in the analysis of the image.

If a fully automated procedure is not available, the base (b) and the height (h) of the drop are measured on the display using a mobile reading scale, then the water contact angles (WCA) are calculated by applying the following formula:

WCA=[arc tg2h/b]×2×180/π

Typically, the values are calculated as a mean of three different measurements taken at room temperature. The precision is usually of about ±5°.

Advantageously, by using the sessile drop method, and considering as reference water contact values of 12° for alpha-lactose monohydrate and of 118° for magnesium stearate, the experimental water contact angle is at least of 34°, more advantageously equal to or higher 36°, preferably equal to or higher than 39°, more preferably equal to or higher than 50°.

By applying the conditions disclosed in the co-pending application EP 10158951.3, which is incorporated herein by reference in its entirety, experimental water contact angles equal to or higher than 90°, preferably higher than 100° may be obtained.

The extent to which the magnesium stearate coats the surface of the lactose particles may also be determined by scanning electron microscopy (SEM), a versatile analytical technique well known in the art.

Such microscopy may be equipped with an EDX analyzer (an Electron Dispersive X-ray analyzer), that can produce an image selective to certain types of atoms, for example magnesium atoms. In this manner it is possible to obtain a clear data set on the distribution of magnesium stearate on the surface of carrier particles.

SEM may alternatively combined with IR or Raman spectroscopy for determining the extent of coating, according to well known procedures.

Another analytical technique that may advantageously be used is X-ray photoelectron spectroscopy (XPS), by which it has been possible to calculate both the extent of coating and the depth of the magnesium sterate film around the lactose particles.

XPS measurements may be taken with commercially available instruments such as Axis-Ultra instrument from Kratos Analytical (Manchester UK), typically using monochromated Al Kα radiation according to known procedures.

The active ingredient is a drug for inhalation bearing a chemical group susceptible to hydrolysis such as a carbonate group, a carbamate group, or an ester group, preferably a carbonate group or a carbamate group, more preferably a carbonate group.

Drugs bearing said groups typically behave as soft drugs and, once inhaled, are degraded by hydrolysis to inactive compounds which are rid of any systemic side effects.

They normally belong to classes which may exhibit undesired side effects due to systemic absorption such as muscarinic receptor antagonists, phosphodiesterase-4 inhibitors, and steroids for inhalation.

Thus, for example, the active ingredient may contain the group (I):

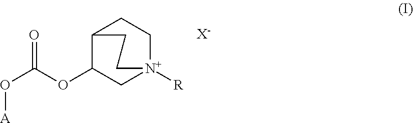

such as the antimuscarinic drugs disclosed in WO 2010/015324, which is incorporated herein by reference in its entirety, wherein:

A may be an optionally substituted aryl or heteroaryl or arylalkyl or heteroarylalkyl or a group of formula (a):

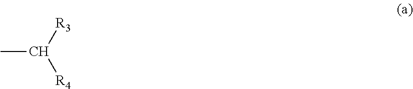

wherein

R₃ and R₄ are the same or different and may be independently selected from the group comprising H, (C₃-C₈)-cycloalkyl, aryl or heteroaryl, wherein said aryl or heteroaryl may be optionally substituted with a halogen atom or with one or several substituents independently selected from the group consisting of —OH, —O—(C₁-C₁₀)-alkyl, oxo (═O), —SH, —S—(C₁-C₁₀)-alkyl, —NO₂, —CN, —CONH₂, —COOH, —(C₁-C₁₀-alkoxycarbonyl, —(C₁-C₁₀)-alkylsulfanyl, —(C₁-C₁₀)-alkylsulfinyl, —(C₁-C₁₀)-alkylsulfonyl, —(C₁-C₁₀)-alkyl, and —(C₁-C₁₀)-alkoxyl, or when R₃ and R₄ are both independently aryl or heteroaryl they may be linked through a Y group which may be a —(CH₂)ₙ—, with n=0-2, wherein when n=0 Y is a single bond, forming a tricyclic ring system wherein the carbon atom of —(CH₂)ₙ— may be substituted by a heteroatom selected from O, S, N and with the proviso that R₃ and R₄ are never both H;

and R is a residue selected from:
—(C₁-C₁₀)-alkyl, —(C₂-C₁₀)-alkenyl, and —(C₂-C₁₀)-alkynyl optionally substituted with a group selected from:
   a group selected from of —NH₂, —NR₁R₂, —CONR₁R₂, —NR₂COR₁, —OH, —SOR₁, —SO₂R₁, —SH, —CN, —NO₂, and alicyclic compounds;
   —Z—R₁, wherein Z is selected from —CO—, —O—, —COO—, —OCO—, —SO₂—, —S—, —SO—, —COS—, and —SCO— or it is a bond; and —(C₃-C₈)-cycloalkyl.

R₁ is a residue selected from:
alicyclic group optionally substituted with one or several substituents independently selected from —OH, oxo (═O), —SH, —NO₂, —CN, —CONH₂, —COOH, —(C₁-C₁₀-alkoxycarbonyl, —(C₁-C₁₀)-alkylsulfanyl, —(C₁-C₁₀)-alkylsulfonyl, —(C₁-C₁₀-alkyl, and —(C₁-C₁₀)-alkoxyl NR₂CO—(C₁-C₁₀)-alkyl;
aryl optionally substituted with —NR₂CO—(C₁-C₁₀)-alkyl, alkyl, —O—(C₁-C₁₀)-alkyl, or halogen and heteroaryl optionally substituted with —NR₂CO—(C₁-C₁₀)-alkyl or halogen.

R₂ is a group selected from H, phenoxycarbonyl, benzyloxycarbonyl, —(C₁-C₁₀)-alkoxycarbonyl, —(C₁-C₁₀)-alkylcarbonyl, —(C₁-C₁₀)-alkylsulfonyl, and —(C₁-C₁₀)-alkyl.

X⁻ is a physiologically acceptable anion such as bromide, chloride, and trifluoroacetate, preferably chloride.

A preferred group of compounds of formula (I) is that wherein R is a methyl substituted by —Z—R₁ group wherein Z is —CO— and R₁ is thienyl, according to formula (II):

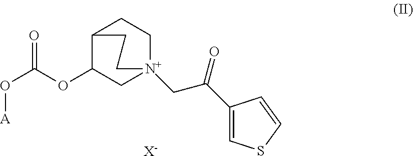

A more preferred group of compounds of formula (II) is that wherein A is a group of formula (a) wherein R₃ and R₄ are both phenyl, optionally substituted with one or more halogen atoms.

Otherwise the active ingredient may contain the group

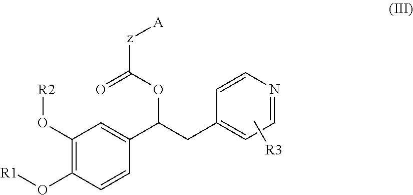

such as carbonate and carbamate phosphodiesterase-4 inhibitors disclosed in the co-pending application n. WO 2009/018909, which is incorporated herein by reference in its entirety, wherein

Z is selected from the group consisting of —O(CH₂)ₚ— wherein p=0, 1, 2 or 3 or —NR₆— wherein R₆ is H or a linear or branched (C₁-C₄) alkyl;

R₁ and R₂ are different or the same and are independently selected from the group consisting of:
H;
linear or branched (C₁-C₆) alkyl, optionally substituted by one or more substituents selected from (C₃-C₇) cycloalkyl or (C₅-C₇) cycloalkenyl;
(C₃-C₇) cycloalkyl;
(C₅-C₇) cycloalkenyl;
linear or branched (C₂-C₆) alkenyl; and
linear or branched (C₂-C₆) alkynyl.

R₃ is one or more substituents independently selected from the group consisting of —H, —CN, —NO₂, —CF₃, and halogen atoms.

A is a ring system, that is a mono- or bicyclic ring which may be saturated, partially unsaturated or unsaturated, such as aryl, (C₃-C₈) cycloalkyl, or heteroaryl, said ring system A having 5 to 10 ring atoms in which at least one ring atom is a heteroatom (e.g. N, S, or O), in which the optional substituent Rₓ on the A ring system may be one or more, may be the same or different, and is independently selected from the group consisting of:
linear or branched (C₁-C₆) alkyl optionally substituted by one or more (C₃-C₇) cycloalkyl;
linear or branched (C₂-C₆) alkenyl optionally substituted by one or more (C₃-C₇) cycloalkyl;
linear or branched (C₂-C₆) alkynyl optionally substituted by one or more (C₃-C₇) cycloalkyl;
(C₅-C₇) cycloalkenyl;
phenyl;
(C₃-C₇) heterocycloalkyl;
OR₇ wherein R₇ is selected from the group consisting of
   —H;
   (C₁-C₁₀) alkyl optionally substituted by one or more (C₃-C₇) cycloalkyl;
   (C₃-C₇) cycloalkyl;
   (C₁-C₄) alkyl-(C₃-C₇) heterocycloalkyl;
   —CO—(C₁-C₆) alkyl;
   —COO—(C₁-C₆) alkyl;
   phenyl;
   benzyl;
   (C₁-C₁₀) alkyl-NR₈R₉ wherein R₈ and R₉ are independently selected from the group consisting of H, linear or branched (C₁-C₆) alkyl and they may form with the nitrogen atom to which they are linked a saturated, partially saturated or unsaturated ring, preferably NR₈R₉ is linked to (C₁-C₁₀) alkyl forming for example saturated, partially saturated or unsaturated piperidine, oxazine, imidazole rings, wherein these rings are optionally substituted by (C₁-C₄) alkyl; and
   halogen atoms;
   —CN;
   —NO₂;
   —NR₁₀R₁₁ wherein R₁₀ and R₁₁ are different or the same and are independently selected from the group consisting of:
      —H;
      linear or branched (C₁-C₆) alkyl, optionally substituted with phenyl or (C₃-C₇) cycloalkyl;
      —COC₆H₅;
      —CO—(C₁-C₄) alkyl;
      —COO—(C₁-C₄) alkyl;
      —CONH—(C₁-C₆) alkyl-R₁₂, wherein R₁₂ is selected from the group consisting of:
         —H;
         (C₁-C₄) alkyl;
         OR₄R₅; and
         —CONH(C₁-C₄) alkyl-N(C₁-C₄) alkyl;
      or they form with the nitrogen atom to which they are linked a saturated or partially saturated ring, preferably a piperidyl ring;
   (C₁-C₄) alkyl-NR₁₀R₁₁;
   —COR₁₂ wherein R₁₂ is phenyl or linear or branched (C₁-C₆) alkyl;
   oxo;
   —HNSO₂R₁₃ wherein R₁₃ is (C₁-C₄) alkyl or a phenyl optionally substituted with halogen atoms or with a (C₁-C₄) alkyl group;
   SO₂R₁₄ wherein R₁₄ is (C₁-C₄) alkyl, OH or NR₁₀R₁₁ wherein R₁₀ and R₁₁ are as defined above;
   —SOR₁₅ wherein R₁₅ is phenyl or (C₁-C₄) alkyl;
   —SR₁₆ wherein R₁₆ is H, phenyl or (C₁-C₄) alkyl;
   —COOR₁₇ wherein R₁₇ is H, (C₁-C₄) alkyl, phenyl or benzyl; and
   —(CH₂)_{q}OR₁₈, wherein q=1, 2, 3, or 4 and R₁₈ is H or (C₁-C₄) cycloalkyl. and pharmaceutically acceptable salts and N-oxides on the pyridine ring thereof.

The active ingredient may also be a steroid for inhalation bearing an ester group of formula (IV):

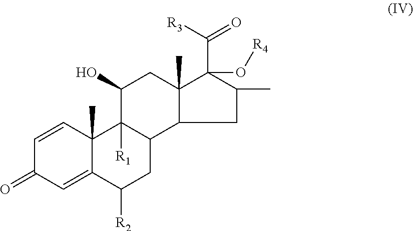

wherein R₁ is —H or a halogen atom selected from the group consisting of F, Cl, Br and I, preferably F or Cl;

R₂ is R₁ is —H or an halogen atom selected from the group consisting of F, Cl, Br and I, preferably H or Cl;

R₃ is a chloromethyl or a fluoromethylthio group; and

R4 is a furoate or a propionate residue.

Preferably, the steroid is fluticasone furoate or propionate or mometasone furoate.

Due to the hydrophobic microenvironment created by the carrier particles coated with magnesium stearate, the chemical stability of said kinds of active ingredients in the formulation during storage is improved.

In particular, very significant results have been obtained with antimuscarinic drugs bearing a carbonate group of general formula (II).

Accordingly, the present invention is also directed to a dry powder pharmaceutical formulation for inhalation comprising (a) carrier particles, (b) magnesium (c) a compound of general formula (II) as active ingredient.

Preferably, magnesium stearate is present in an amount of 0.1 to 1.0% based on the total weight of the carrier and coats the surface of the carrier particles.

The carrier particles are preferably made of alpha-lactose monohydrate and have a particle size of 212 to 355 microns.

In order that the active ingredient is inhalable, i.e. it can pass into the deep lung such as the terminal and respiratory bronchioles and the alveolar ducts and sacs, it must be in particulate form having a mean particle diameter (measured as the mass mean diameter) of at most about 10 microns, preferably 1 to 6 microns, more preferably 2 to 4 microns. Such microfine particles can be obtained in a manner known per se, for example by micronization, controlled precipitation from selected solvents, or by spray drying.

The compound of formula (II) is typically present in an amount of 0.01 to 5%, w/w, based on the total weight of the composition, preferably 0.05 to 4% w/w, more preferably 0.1 to 2.5% w/w, based on the total weight of the composition.

The full single therapeutically effective dose (hereinafter the single dose) of compound of formula (II) is advantageously 1 in to 1000 μg, more advantageously 5 μg to 800 μg, preferably 20 to 500 μg, more preferably 200 to 350 μg.

Said single dose will depend on the kind and the severity of the disease and the conditions (weight, sex, age) of the patient and shall be administered one or more times a day.

The dry powder formulations of the invention could also comprise further active ingredients such as drugs belonging to the classes of steroids for inhalation, beta2-agonists, and phosphopiesterase-4 inhibitors, and their combinations.

The formulation of the invention can be prepared according to known methods, and may be utilized with any dry powder inhaler.

Dry powder inhalers can be divided into two basic types:

i) single dose inhalers, for the administration of single subdivided doses of the active compound; each single dose is usually filled in a capsule; and

ii) multidose inhalers pre-loaded with quantities of active principles sufficient for longer treatment cycles.

The dry powder formulation for inhalation according to the present invention is particularly suitable for multidose dry powder inhalers comprising a reservoir from which individual therapeutic dosages can be withdrawn on demand through actuation of the device, for example that described in WO 2004/012801, which is incorporated herein by reference in its entirety.

Administration of the formulations of the present invention may be indicated for prevention and/or the treatment of mild, moderate or severe acute or chronic symptoms or for prophylactic treatment of an inflammatory or obstructive airways disease such as asthma and chronic obstructive pulmonary disease (COPD). Other respiratory disorders characterized by obstruction of the peripheral airways as a result of inflammation and presence of mucus may also benefit from the formulation of the invention.

Other features of the invention will become apparent in the course of the following descriptions of exemplary embodiments which are given for illustration of the invention and are not intended to be limiting thereof.

### Examples

The investigation was aimed at evaluating the chemical compatibility of active ingredients bearing a group susceptible to hydrolysis with carriers for powders for inhalation.

### Test Compound.

The drug used as the test compound was (R)-3-[bis-(3-fluoro-phenyl)-methoxycarbonyloxy]-1-(2-oxo-2-thiophen-2-yl-ethyl)-1-azonia-bicyclo[2.2.2]octane; chloride. This drug and its synthesis are disclosed in WO 2009/015324, which is incorporated herein by reference in its entirety. It was micronized before use leading to a particle size of d(v, 0.1)=0.7 μm, d(v, 0.5)=2.7 μm, d(v, 0.9)=9.2 μm.

### Excipients.

Alpha-lactose monohydrate commercially available as Capsulac® 60 (Meggle) was used. The fraction of interest, 212-355 microns was obtained by sieving. Magnesium stearate of vegetable origin was used having a starting particle size with a MMD of less than 10 microns.

### Formulations.

Dry powder formulations were prepared comprising as a carrier either particles of alpha-lactose monohydrate 212-355 microns alone (coarse) or coated with magnesium stearate (coated). Said coated particles were obtained by mixing alpha-lactose monohydrate with 1% magnesium stearate in a Turbula mixer for 4 hours. The unit formulae are reported in Table 1.

TABLE 1

| Strength | Coarse (20 μg/20 mg) | Coated (20 μg/20 mg) |
|---|---|---|
| Lactose 212-355 μ | 19.98 mg | 19.77 |
| Magnesium stearate 1% | — | 0.21 |
| Test compound | 0.020 mg | 0.02 mg |
| Total | 20 mg | 20 mg |

Samples of the manufactured mixtures were filled in glass vials and stored under stressing conditions (90° C., 60% relative humidity and 90° C., 75% relative humidity) in comparison to a sample stored under long term conditions (25° C., 60% relative humidity).

The percentage amount of residual test compound and of the degradation product due to hydrolysis, (R)-1-hydroxy-(2-oxo-2-thiophen-2-yl-ethyl)-1-azonia-bicyclo[2.2.2]octane; chloride quoted as CHF 5551.02, was determined in the two formulations by ultra performance liquid chromatography (UPLC) in comparison to the active ingredient without the carrier (API). The results are reported in Tables 2 and 3. The variation of CHF 5551.2 content with the time is also plotted in FIG. 3.

TABLE 2

| | | Carrier | | | | |
|---|---|---|---|---|---|---|
| | | — | Coarse | Coarse | Coated | Coated |
| | Temperature | 25 | 90 | 90 | 90 | 90 |
| | (° C.) | | | | | |
| | Rel. humidity | 60 | 60 | 75 | 60 | 75 |
| | (%) | | | | | |
| Check | | | | | | |
| point no. | Time (days) | Test compound values (%) | | | | |
| 0 | 0 | 100.0 | 100.0 | 100.0 | 100.0 | 100.0 |
| 1 | 2 | 99.9 | 99.9 | 100.0 | 100.0 | 100.0 |
| 2 | 4 | 99.9 | 99.8 | 99.8 | 99.98 | 99.9 |
| 3 | 7 | 99.9 | 99.7 | 99.5 | 99.8 | 99.6 |
| 4 | 9 | 99.9 | 99.6 | 99.5 | 99.8 | 99.6 |
| 5 | 11 | 99.9 | 99.5 | 99.4 | 99.8 | 99.6 |
| 6 | 14 | 100.0 | 99.3 | 99.3 | 99.8 | 99.5 |

TABLE 3

| | | Carrier | | | | |
|---|---|---|---|---|---|---|
| | | — | Coarse | Coarse | Coated | Coated |
| | Temperature | 25 | 90 | 90 | 90 | 90 |
| | (° C.) | | | | | |
| | Rel. humidity | 60 | 60 | 75 | 60 | 75 |
| | (%) | | | | | |
| Check | | | | | | |
| point no. | Time (days) | CHF 5551.02 values (%) | | | | |
| 0 | 0 | 0.08 | 0.08 | 0.08 | 0.07 | 0.07 |
| 1 | 2 | 0.20 | 0.27 | 0.25 | 0.24 | 0.26 |
| 2 | 4 | 0.29 | 0.38 | 0.43 | 0.39 | 0.37 |
| 3 | 7 | 0.24 | 0.56 | 0.66 | 0.44 | 0.63 |
| 4 | 9 | 0.28 | 0.56 | 0.74 | 0.47 | 0.66 |
| 5 | 11 | 0.29 | 0.74 | 0.81 | 0.51 | 0.66 |
| 6 | 14 | 0.23 | 0.90 | 0.87 | 0.52 | 0.80 |

The results indicate that the amount of the degradation product due to hydrolysis increases moving from a carrier made of only alpha-lactose monohydrate to the coated particles.

Where a numerical limit or range is stated herein, the endpoints are included. Also, all values and subranges within a numerical limit or range are specifically included as if explicitly written out.

Obviously, numerous modifications and variations of the present invention are possible in light of the above teachings. It is therefore to be understood that, within the scope of the appended claims, the invention may be practiced otherwise than as specifically described herein.

All patents and other references mentioned above are incorporated in full herein by this reference, the same as if set forth at length.

1-17. (canceled)

18. A method of inhibiting or reducing chemical degradation of an anti-muscarinic active ingredient in a powder formulation comprising carrier particles and the anti-muscarinic active ingredient, the method comprising:
coating a surface of the carrier particles with magnesium stearate in preparation of the powder formulation;
wherein:
the anti-muscarinic active ingredient is a compound susceptible to hydrolysis and bearing an ester or a carbonate functional group;
the carrier particles have a mass diameter of 150 to 400 microns;
after coating, a percentage of surface coating of the carrier particles by magnesium stearate is at least 20%;
the coated carrier particles comprise magnesium stearate in an amount of 0.1 to 1.0% by weight; and
after storing the powder formulation for 14 days at a temperature of 90° C. and a relative humidity of 75%, a hydrolysis product formed by hydrolysis of the anti-muscarinic active ingredient is formed in an amount of less than 0.8% by weight based on a total weight of the powder formulation.

19. The method according to claim 18, wherein the carrier particles have a mass diameter of 210 to 355 microns.

20. The method according to claim 18, wherein the carrier particles comprise a crystalline sugar.

21. The method according to claim 19, wherein the carrier particles comprise a crystalline sugar.

22. The method according to claim 20, wherein the crystalline sugar is alpha-lactose monohydrate.

23. The method according to claim 18, wherein the coated carrier particles comprise magnesium stearate in an amount of 0.15 to 0.5% by weight.

24. The method according to claim 19, wherein the coated carrier particles comprise magnesium stearate in an amount of 0.15 to 0.5% by weight.

25. The method according to claim 20, wherein the coated carrier particles comprise magnesium stearate in an amount of 0.15 to 0.5% by weight.

26. The method according to claim 22, wherein the coated carrier particles comprise magnesium stearate in an amount of 0.15 to 0.5% by weight.

27. The method according to claim 22, wherein the coated carrier particles comprise magnesium stearate in an amount of 0.2 to 0.4% by weight.

28. The method according to claim 18, wherein after coating, the percentage of surface coating of the carrier particles by magnesium stearate is at least 60%.

29. The method according to claim 28, wherein after coating, the percentage of surface coating of the carrier particles by magnesium stearate is at least 70%.

30. The method according to claim 29, wherein after coating, the percentage of surface coating of the carrier particles by magnesium stearate is at least 80%.

31. The method according to claim 30, wherein after coating, the percentage of surface coating of the carrier particles by magnesium stearate is at least 90%.
